(19) United States
(12) Patent Application Publication
(10) Pub. No.: US 2007/0244101 A1
(43) Pub. Date: Oct. 18, 2007

(54) NOVEL COMPOSITIONS AND METHODS OF TREATMENT OF TYPE II DIABETES AND HYPERTENSION

(76) Inventor: Lawrence Solomon, Boca Raton, FL (US)

Correspondence Address: ACCU-BREAK TECHNOLOGIES, INC., 1000 SOUTH PINE ISLAND ROAD, SUITE 230, PLANTATION, FL, 33324, US

(21) Appl. No.: 11/736,346

(22) Filed: Apr. 17, 2007

Related U.S. Application Data
(60) Provisional application No. 60/792,590, filed on Apr. 17, 2006.

(51) Int. Cl. A61K 31/545; A61K 31/4439; A61K 31/426

(52) U.S. Cl. 514/223.5; 514/342; 514/369

(57) ABSTRACT

The subject invention relates to a composition and method for treating a patient employing co-formulation or co-administration of a thiazolidinedione (TZD) and a diuretic.

### CROSS-REFERENCE TO RELATED APPLICATIONS

This is a continuation-in-part of U.S. Provisional Appl'n Ser. No. 60/792,590, filed Apr. 17, 2006.

### BACKGROUND OF THE INVENTION

It is known that thiazolidinediones (TZDs), such as pioglitiazone and rosiglitazone, lower blood sugar in type II diabetes mellitus and induce fluid retention, or edema, in many patients. A current article entitled “Do thiazolidinediones cause heart failure? A critical review,” by Tang. W. H., in Cleveland Clinic Journal of Medicine, April 2006 (pages 390-97), recommends that for relief of the edema, withdrawal of the TZD may be the only option, although “loop diuretics help to some degree.” Also disclosed by Tang is the addition of spironolactone and reducing the TZD dosage. An angiotensin-converting enzyme inhibitor (“ACEI”) with or without a thiazide diuretic, may also be effective to reduce edema, according to Tang.

In “Thiazolidinedione-Associated Congestive Heart Failure and Pulmonary Edema”, Kermani and Garg, Mayo Clinic Proceedings, 2003, Vol. 78:2088-91, it was reported that cases of pulmonary edema were all treated with furosemide, a loop diuretic. None of those cases reported treatment with only a thiazide diuretic or initiating treatment with a thiazide diuretic.

The label (Product Information; “PI”) for pioglitazone (Actos®) and for rosiglitazone (Avandia®) do not recommend diuretic treatment for prevention or treatment of edema due to those drugs, though the PI's for both drug products state that edema is induced in patients taking the TZDs at a rate greater than placebo.

Another type of well known product is thiazide diuretics, such a hydrochlorothiazide (HCTZ) and many others. Typically grouped with thiazides is the “thiazide-type” diuretic (“TTD”), chlorthalidone, which is chemically derived form a thiazide, but has a different ring structure. The thiazide-type diuretics are generally indicated for hypertension treatment and while they are often indicated for edema treatment, it is not known whether they are effective in either treating or preventing the occurrence of TZD-induced edema or preventing the worsening of edema in TZD-treated patients with pre-existing edema. The Tang article conspicuously fails to recommend thiazides for treatment of TZD-induced edema, except as incidental to the use of an ACEI.

Recent retrospective reports have analyzed data from TZD treatment studies and concluded that TZDs lower blood pressure (BP) modestly but statistically significantly. Whether this proposed BP lowering is an effective treatment for hypertension or whether this proposed BP lowering would be effective with concomitant use of a TTD, is not known.

No recommendation to treat with a diuretic, or more specifically with a TTD, before the onset of edema caused by initiation of TZD therapy or dose increase of a TZD has previously been made.

In addition, no suggestion has been made that, in treating hypertension with a TZD, a TZD should be given prophylactically in an otherwise controlled diabetic patient or in a pre-diabetic patient, either concomitantly with or around the same time as initiation of treatment with a TTD, or with dose escalation of a thiazide.

There has been no known recommendation to formulate a TZD and a TTD. There has been no known recommendation to routinely co-administer a TZD and a TTD, or to co-administer a TZD and a TTD at all, except in the presence of an ACEI.

### SUMMARY OF THE INVENTION

The subject invention comprises, in one embodiment, a co-formulation of a TZD and a thiazide-type diuretic in a single dosage form. Various unexpected benefits can result from such co-formulation. Also included within the invention are the co-formulation in a single dosage form of a TZD and a loop diuretic, the loop diuretic being either at a natriuretic dose or a non-natriuretic dose. The subject invention further comprises a combination of a potassium-retaining (“potassium-sparing”) diuretic with a TZD in a single dosage form. Potassium-retaining diuretics of the subject invention include antihypertensives, such as spironolactone, or non-antihypertensives, such as triamterene.

In another embodiment of the subject invention, a TZD and a diuretic, such as a thiazide diuretic, a thiazide-type diuretic, a loop diuretic, or a potassium-retaining diuretic can be provided each as a separate dosage form and co-packaged to provide a combination treatment for diabetes and the edema associated with the administration of the TZD. The method of the subject invention also comprises co-administration of the TZD and diuretic components, whether administered as a co-formulation of both drug components in a single dosage form or co-administered as separate dosage forms.

The invention also encompasses novel uses of the of the TZD and diuretic drugs with each other. Uniquely, the invention discloses a combination product in which each component offsets an unrelated side effect of the other: diabetes (and also cholesterol elevation) caused by the diuretic, and edema caused by the TZD. This is different from a typical case of side effect reduction in combination products as in Lotrel®, in which benazepril offsets the edema caused by amlodipine, or the other common case such as in combination products in which one drug is a potassium-wasting diuretic and the other drug induces potassium retention (including drugs such as potassium-retaining diuretics, beta-blockers, and angiotensin-receptor blocking agents), so that the same parameter is affected in opposite directions by the different agents.

Furthermore, the invention also uniquely provides additive benefit, in that both the thiazide and the TZD individually can lower BP.

### DETAILED DESCRIPTION OF THE INVENTION

Thiazide-type diuretics include HCTZ, chlorothiazide, chlorthalidone, and many others as are found in standard textbooks such as Goodman and Gilman's The Pharmacological Basis of Therapeutics (2006) 11^{th} Ed. Included for discussion and inventive purposes herein are other diuretics such as indapamide that are often not considered to be TTDs.

Loop diuretics useful in the compositions and method of the subject invention include without limitation furosemide, torsemide, bumetamide, ethacrynic acid, and the like.

TZDs include with limitation piogliatazone, rosiglitazone, troglitazone, and the like.

Doses used comprise those known in the art to be safe and effective for their approved purpose. In addition, it would be recognized that the active ingredient used in the compositions and methods of the subject invention can include the use of a pharmaceutical salt, active metabolite, prodrug, derivative, polymorph or hydrate of the active ingredient, or a combination or mixture thereof.

Another benefit of the invention involves preventing the onset of diabetes in a patient, such as one with pre-diabetes, which is variously defined quantitatively as blood sugar in the 100-200 range or in dictionaries as, for example, an asymptomatic abnormal state that precedes the development of clinically evident diabetes (Merriam-Webster's Medical Dictionary, 2002).

Formulations of the invention include the use of known pharmaceutically acceptable excipients as would be readily understood in the art in view of the subject disclosure. Such excipients and carriers are described, for example, in “Remington's Pharmaceutical Sciences” Mack Pub. Co., New Jersey (1991), which is incorporated herein by reference.

It is recognized that related inventions may be within the spirit of the disclosures herein. Also no omission in the current application is intended to limit the inventors to the current claims or disclosures. While certain preferred and alternative embodiments of the invention have been set forth for purposes of disclosing the invention, modifications to the disclosed embodiments may occur to those who are skilled in the art.

1. A pharmaceutical composition or co-packaging comprising a TZD plus a diuretic selected from the group consisting of a thiazide-type diuretic (“TTD”), a loop diuretic, and a potassium-retaining diuretic.

2. A method of treatment comprising administering within the same day a TZD plus a diuretic.

3. The method of treatment to claim 2, wherein said TZD and diuretic are administered within about 12 hours of each other.

4. The method of claim 2 wherein said TZD and diuretic are administered within about 4 hours of each other.

5. The method of claim 2 wherein said TZD and diuretic are administered within about 1 hour of each other.

6. The method of claim 2 wherein said TZD and diuretic are administered concomitantly.

7. The method of claim 6 wherein said TZD and diuretic are concomitantly administered within a single dosage form.

8. The composition as in claim 1, in which the TZD and the diuretic are additive with respect to blood pressure lowering in a hypertensive patient.

9. The composition of claim 1 wherein said TZD and diuretic are synergistic with regard to blood pressure lowering in a patient suffering from hypertension.

10. The composition of claim 1 wherein neither of said TZD and diuretic has a statistically significant (demonstrable) BP benefit vs. placebo but the combination effects a statistically significant BP benefit.

11. The composition of claim 1, wherein the TZD lacks a statistically significant or otherwise demonstrable benefit vs. placebo and the diuretic is statistically significantly better than placebo, and the combination demonstrates a benefit of the combination vs. the diuretic, the parameter referred to in this claim being blood pressure lowering in a patient suffering from hypertension.

12. The composition of claim 1, wherein the diuretic lacks a statistically significant or otherwise demonstrable benefit vs. placebo and the TZD is statistically significantly better than placebo, and the combination demonstrates a benefit of the combination vs. the TZD, the parameter being referred to in this claim being blood pressure lowering in a patient suffering from hypertension.

13. A pharmaceutical composition comprising at least two active drug components in which the at least two components each offset or mitigate a side effect of the other component, involving other than the situation in which each drug has opposing actions on the same parameter.

14. A method of treating a patient with a diuretic to prevent either the onset of edema, the worsening of edema, the onset of congestive heart failure, or the worsening of congestive heart failure, by treating with said diuretic a patient receiving or scheduled to being to receive TZD.

15. A method of treating edema caused or worsened by a TZD by concomitant administration of a TTD with said TZD, either in separate dosage forms or in a co-formulation.

16. A method of preventing the onset of diabetes mellitus (“diabetes”) in patients with pre-diabetes, said method comprising administering a TZD in said patient.

17. The method of claim 16 wherein said patient is further administered a diuretic.

18. The method of claim 16 wherein said patient is receiving or scheduled (about) to receive a diuretic.

19. A method of preventing the worsening of glycemic status in a patient scheduled (about) to receive a diuretic by co-administering a TZD, said method comprising administering a diuretic.

20. A method of preventing or ameliorating a rise in serum cholesterol due to a diuretic by co-administration of a TZD.

21. The composition of claim 1 wherein said TZD is pioglitazone.

22. The composition of claim 1 wherein said diuretic is HCTZ or chlorthalidone.
